(19) United States
(12) Patent Application Publication
(10) Pub. No.: US 2016/0208209 A1
(43) Pub. Date: Jul. 21, 2016

(54) Bioreactor Docking Station Systems, and Methods of Use Thereof

(71) Applicant: Dakota Systems, Inc., Dracut, MA (US)

(72) Inventors: John M. Thomas, North Andover, MA (US); Stephen M. Perreault, East Kingston, NH (US)

(21) Appl. No.: 14/992,303

(22) Filed: Jan. 11, 2016

Related U.S. Application Data
(60) Provisional application No. 62/104,901, filed on Jan. 19, 2015.

(51) Int. Cl. C12M 1/12; B01L 1/00; C12M 1/34; C12M 3/00; C12M 1/00; C12M 1/02

(52) U.S. Cl. CPC C12M 37/00; C12M 23/52; C12M 23/50; C12M 41/18; C12M 41/44; C12M 27/00; C12M 23/40; C12M 23/48; B01L 1/00; B01L 2200/141

(57) ABSTRACT

A system and method for docking and using bioreactors. There is a sterile suite that is constructed and arranged to house one or more bioreactors and a human-machine interface, and a separate utility space that is not sterile, and that is constructed and arranged to house sources of electricity, liquid and gas. There is a clean-room wall separating the sterile suite from the utility space. A docking station is located in the clean-room wall, with part of it (e.g., one side) in the sterile suite and another part of it (e.g., another side) in the utility space. The docking station has electrical, liquid and gas connections that the bioreactor can connect to on the sterile suite side of the docking station, and that the sources of electricity, liquid and gas can connect to on the utility space side of the docking station.

### CROSS-REFERENCE TO RELATED APPLICATION

This application claims priority of Provisional Patent Application 62/104,901, filed on Jan. 19, 2015.

### BACKGROUND

This disclosure relates to bioreactors.

At its inception cell culture manufacturing relied on custom stainless steel bioreactors, requiring large steam plants and WFI (water for injection) water plants, to perform the cell culture and fermentation processes. The bioreactors were built as unique units that were costly to maintain and operate. They required expensive to operate plant wide utilities, which had to be large in order to cover peak use and had a large downtime between batches. Although expensive to operate the stainless steel bioreactors did not leach any material into the cell culture media and could be sterilized with 100% surety with steam.

Subsequently, patents were issued for disposable bag bioreactors (such as U.S. Pat. No. 7,629,167 B2) that eliminated the need for sterilization steam since the bioreactor bag is disposable after use. Therefore the steam plants and “Clean in Place” (CIP) skids were no longer needed as a new supposedly sterile bag is used for each product batch with the old one from the previous batch disposed of as medical waste. Time between batches was reduced as the old bag was disposed of and a new bag used at the start of the new batch.

Over time it became to be noticed that the cytotoxic compound bDtBPP [bis(2,4-di-tert-butylphenyl)phosphate], from the plastic material in the bag, can leach into the cell culture media during human protein production via mammalian cells. This contaminates the cell culture media and can inhibit cell growth at concentrations as low as 100 ug/l. It appears the ionizing radiation used to sterilize these bags creates the bDtBPP. bDtBPP accumulates in the cell media over time and contact of the media with the bag of only a few days can easily accumulate enough bDtBPP to inhibit cell growth and lower cell counts.

Disposable bag systems were developed to eliminate steam and CIP requirements as well as to reduce time between batches. But with the discovery that the practice of sterilizing these bags can release a cytotoxic compound into the media, it throws the use of single bag systems into question. Unknown future discoveries of leachables raise further questions about the purity of drugs going to the general public. Leachables have been discovered in containers used for drugs as well as in cardiopulmonary bypass machines where the leachables from the PVC tubing cause the blood flowing through the tubing to have a systematic inflammatory response (SIRS), causing swelling and fever in some patients. Leachables are a serious problem in all phases of the pharmaceutical industry.

### SUMMARY

The systems and methods disclosed herein maintain the benefits of reduced downtime and low utility use that bag reactors supply, but uses the original non-leachable, easily sterlizable stainless steel vessels in place of the leachable bags. End users who have already changed over to bag reactors and no longer have steam plants can use the subject system as a means to return to stainless steel vessels/systems. The system comprises a docking station (also called a facility panel), with utilities that can be sized over a range of bioreactor vessels. The vessels can be operably connected to the docking station, one at a time. Any vessel within the size range that is supported by a docking station can be docked and plugged into the utilities that are present at the docking station. Standard bioreactor size ranges that could be supported by a single docking station could be, for example, 100 L(liters)-500 L; 500 L to 1000 L; and 1000 L to 2000 L.

Cell culture requires scale up in batch sizes from the initial beaker in the lab to pilot scale bioreactors. During scale up, vessel sizes need to be close to the same h/d ratios which give typical scale up vessel sizes of 100 L, 250 L, 500 L, 1000 L, 2000 L, and 5000 L. A single docking station can handle all the scale-up vessels in the pilot range up to and including 500 liters, although a second docking station would likely be needed to keep the batch viable during transfer between vessels. Lately the trend is for smaller batches so a small contract manufacturer could run through the scale up procedure using a single docking station, with a second free docking station as long as the maximum batch is 500 L. The current art is for each stainless steel bioreactor to have its own utility set-up. With the subject system, vessels can be configured for cell culture or fermentation and can have different agitators or baffles installed which give more flexibility to the end user.

Vessel temperature control in the system can be designed such that it does not use plant steam, but instead uses a properly sized temperature control unit. This eliminates the current art of having large plant-wide steam generators that run continually regardless of the actual load. In the present system, the smaller clean steam generator(s) are only run during sterilization, which saves utility costs and can save capital costs as well. A mini clean steam generator can be used to supply clean steam condensate to the mechanical seals. The mechanical seals are the only item that needs a continuous supply of clean steam and operating costs are substancially lower using a very small unit to supply this need.

Featured in this disclosure is a bioreactor utility docking station that is constructed and arranged so as to accept (i.e., be operably coupled to) a range of bioreactor vessel sizes. This allows a facility to have one common utility set-up that can handle the range of vessels which would plug into the docking station. The utilities are sized to accommodate the range of vessels. The utilities are located in a separate dirty/utility area rather than in the clean room where the bioreactor(s) are located. This is a complete solution for the end user with the end user only having to supply: city water, WFI water, a bio-waste drain, a single electrical power input, and the gases used in the process.

### BRIEF DESCRIPTION OF THE DRAWINGS

Other objects, features and advantages will occur to those skilled in the art from the following description and the accompanying drawings, in which:

FIG. 1 is a schematic diagram of a bioreactor docking station system.

FIG. 2 is a schematic diagram of a bioreactor skid.

FIG. 3 is a schematic diagram of another bioreactor docking station system.

### DETAILED DESCRIPTION

An advantage of the subject utility docking station, system and method is that the traditional set-up of each vessel having its own utilities on board the vessel skid is no longer necessary. This way the facility need only have one utility set-up per the range of vessels, allowing the facility to easily change between vessel/batch sizes by just disconnecting one vessel and plugging in another. This is a solution that is ideally suited for processes that don't run continuously throughout the year, and allows a common utility to be used for various processes and drug development (such as contract manufacturers/R&D/small batch runs). It also uses a stainless steel bioreactor that does not have the problem of leachables as in a single use bag bioreactor.

Non-limiting examples of the subject system can be described divided into three sections, as follows.

### 1. Docking Station

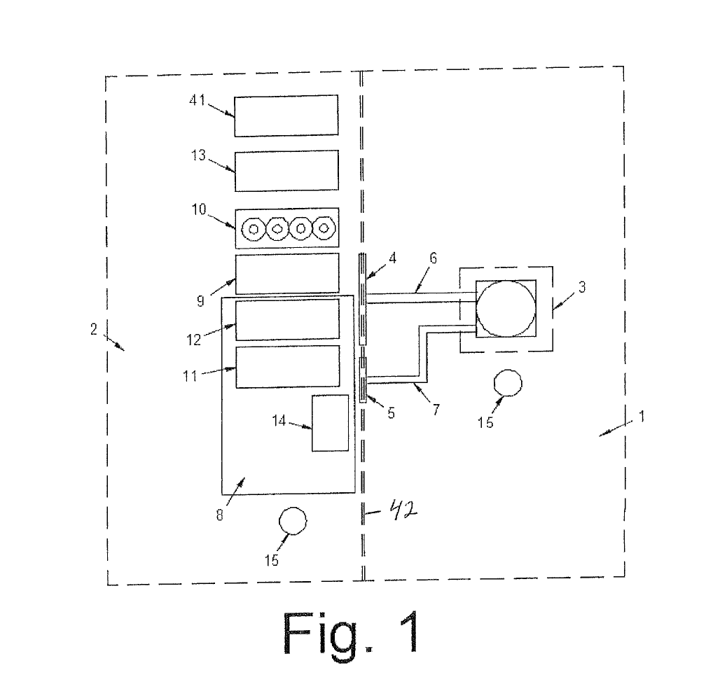

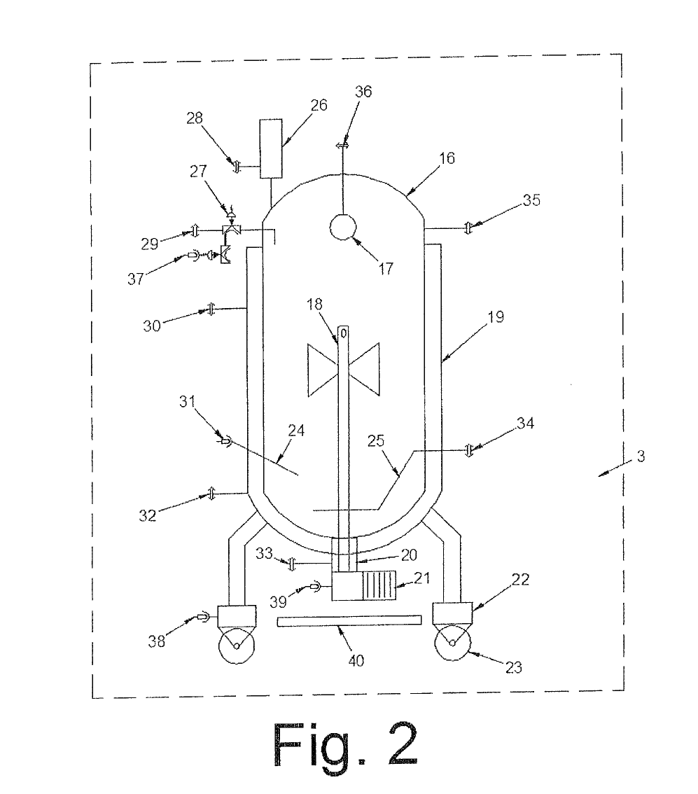

The docking station (4) protrudes into the clean space (sterile suite) (1) and serves as the connection point between a separate non-sterile utility space (2) and the clean space (1) as shown in FIG. 1. Clean room wall (42) separates sterile suite (1) from utility space (2). Docking station (4) has electrical connections (5) which can consist of plugs for power and connections for signals. There can be a HMI (human-machine interface), not shown, to enable the operator to operate the bioreactor system. The docking station (4) has piping connections that can consist of sparge gas (Air, O2, CO2, N2), overlay gas (Air), exhaust gases, vessel temperature control jacket water supply and return, clean steam to the mechanical seals on the bioreactor (20) as shown in FIG. 2, and a transfer panel that supplies either clean steam or CIP solution to the bioreactor through a common manifold for sterilization and cleaning respectively. Flex hoses (6) are used to connect the piping connections on the docking station to the bioreactor skid (3). An electrical cable (7) is used to connect power to the bioreactor skid (3).

### 2. Facility Skid/Chase Area

The facility skid (8) is located in the chase/utility area (2) of the plant. It houses the electrical and pneumatic controls (14), the jacket tempered water skid (12), and the mini clean steam generator (11) that supplies clean steam condensate to the seals. The mini clean steam generator is sized only enough to supply the required clean steam condensate to the mechanical seals and due to its small size, is very economical to operate. This eliminates the need for a larger steam unit to operate continuously as during normal operation, only the mechanical seals need clean steam. Optionally, the facility skid can include a change over system (9) for the gas bottles, support frame for the gas bottles (10), a moveable properly sized electric clean steam generator (13) and a movable clean in place (CIP) skid (41). The end user will need to supply city water, WFI water, electrical power and the gases used in the process. The user will also supply a bio-waste drain (15) in sterile suite (1) and another drain (15) in chase/utility area (2). The optional change over system (9) can be used to change to a full gas bottle when the current bottle is empty. Either bottles or bulk supply can be used by the end user. The facility skid (8) can house mass flow controllers to control the flow of gases to the bioreactor skid (3), with the HMI used to control settings.

Since the components are in a chase/utility area, non-wash down rated components can be used, saving the end user significant money. The facility skid (8) can be sized to accommodate a range of bioreactor vessels 100 L-500 L, 500 L-1000 L, 1000 L-2000 L. Any bioreactor vessel (3) can be docked to the docking station (4) so as to make connections to the facility skid (8), as long as the vessel is within the range of sizes that is supported by the facility skid. Both standard and optional features are shown in FIG. 1. Since the clean steam generator (13) and the CIP skid (41) are moveable, they can be moved to docking stations that need them, in the same utility room or a different utility room. Clean steam and CIP can be used for sterilization and cleaning, respectively, at the very beginning of the cell culture batch and then are not needed for the majority of the batch. Properly sized CIP and clean steam skids can be moved between multiple facility skids as needed.

### 3. Bioreactor Skid

A typical bioreactor skid (3) is shown in FIG. 2. It is placed in the clean manufacturing area (1). It is on wheels (23) and provided with load cells (22) used for determining vessel (16) volume by weight. The bioreactor skid (3) can have a mechanical seal (20), temperature control jacket (19), condensate manifold (40), agitator (18), agitator motor (21), exhaust vent filter (26), spray ball (17), gas sparger (25), temperature probe (24) and addition valves (27). The electrical umbilical cord can contain probe signals (31), load cell signals (38), pneumatic valve connection (37) and power to the agitator motor (21) via a high voltage plug (39). Piping connections can include exhaust gas (28), addition port (29), jacket inlet (32), jacket outlet (30), clean steam to the seals (33), clean steam or CIP solution to the spray balls (36) sparge gas (34) and overlay gas (35). The mechanical seals (20) are designed for minimal maintenance and a time between maintenance of 2-3 years on the seals after continuous operation is not unusual. The bioreactor skid (3) can be rolled to the docking station (4) and the connections made for operation. Other options for components, piping or features may be present as known by those skilled in the art.

Example of Docking Station Use

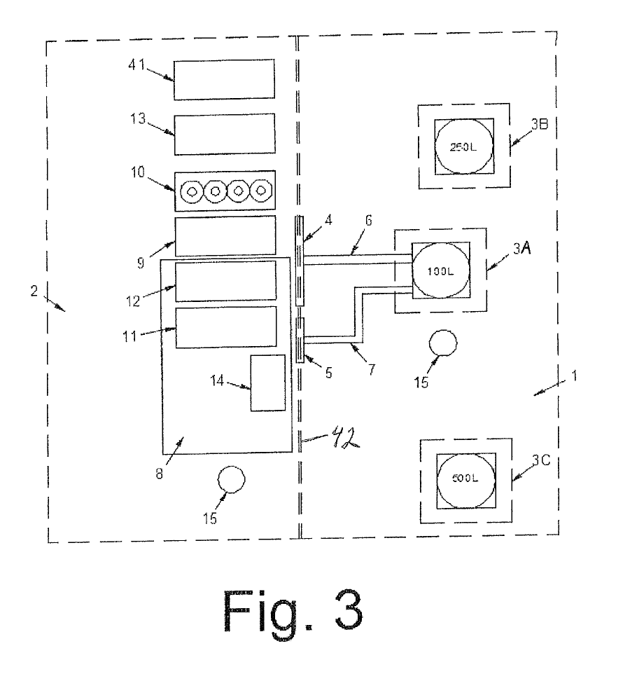

FIG. 3 shows a 100 L vessel (3A) attached to the facility skid and running a cell culture run, with a cleaned and sterilized 250 L vessel (3B) plus a cleaned and sterilized 500 L vessel (3C) waiting. When the cell density gets to the proper level in the 100 L vessel (3A), it will be harvested and transferred to the 250 L vessel (3B), which in the meantime has been connected to another docking station (not shown). More media would be added and when the cell density in the 250 L (3B) gets to the proper level it will be harvested and transferred to the 500 L vessel (3C), which in the meantime has been connected to another docking station (not shown). More media would again be added and the process would continue. When cell density get to the proper level in the 500 L it will be harvested and could either move on to downstream purification or over to a larger 1000 L vessel. While this process is going on, the previous vessels that were used in the process would be cleaned and sterilized to be ready for docking to the facility skid. With smaller contract manufacturers, the final run before purification would be 500 L or even 250 L allowing the whole process to be run with one facility set-up, but at least two properly sized docking stations would in this case need to be present to protect the cell batch. One station is needed for the completed batch, and one station for the vessel to be transferred to. One, more than one, or all of the docking stations can be located in wall 42 so that they are accessible to both the utility room and the clean room.

A number of implementations have been described. Nevertheless, it will be understood that additional modifications may be made without departing from the scope of the inventive concepts described herein, and, accordingly, other embodiments are within the scope of the following claims.

What is claimed is:

1. A system, comprising:
a sterile suite that is constructed and arranged to house one or more bioreactors;
a separate utility space that is not sterile, and that is constructed and arranged to house sources of electricity, liquid and gas;
a clean-room wall separating the sterile suite from the utility space; and
a first docking station located in the clean-room wall and with one side in the sterile suite and another side in the utility space, the first docking station comprising electrical, liquid and gas connections that a bioreactor can connect to on the sterile suite side of the first docking station, and that the sources of electricity, liquid and gas can connect to on the utility space side of the first docking station.

2. The system of claim 1 wherein the bioreactors are on wheeled skids.

3. The system of claim 1 further comprising at least one of the following pieces of equipment in the utility area: a jacket tempered water skid, a gas bottle change-over system, a support frame for gas bottles, a movable electric clean steam generator, a movable clean in place skid, and mass flow controllers.

4. The system of claim 1 further comprising a second docking station located in the clean-room wall and with one side in the sterile suite and another side in the utility space, the second docking station comprising electrical, liquid and gas connections that a bioreactor can connect to on the sterile suite side of the second docking station, and that the sources of electricity, liquid and gas can connect to on the utility space side of the second docking station.

5. The system of claim 4 comprising at least two bioreactors in the sterile suite and comprising bioreactors having different volumes.

6. The system of claim 5 wherein the bioreactors are in one or more of the following volume ranges: 100-500 liters, 500-1000 liters, and 1000-2000 liters.

7. The system of claim 1 wherein the docking station comprises piping connections that can consist of sparge gas (Air, O2, CO2, N2), overlay gas (Air), exhaust gases, vessel temperature control jacket water supply and return, and clean steam to the mechanical seals on a bioreactor using a mini clean steam generator located in the utility space.

8. The system of claim 7 wherein the docking station comprises a transfer panel that supplies either clean steam or CIP solution to the bioreactor through a common manifold.

9. The system of claim 7 wherein flex hoses are used to connect the piping connections on the docking station to the bioreactor and an electrical cable is used to connect power to the bioreactor.

10. The system of claim 1 further comprising a facility skid located in the utility area.

11. The system of claim 10 wherein the facility skid houses electrical and pneumatic controls.

12. The system of claim 11 wherein the utility area further comprises a jacket tempered water skid.

13. The system of claim 12 wherein the utility area further comprises an electric clean steam generator for sterilization and an electric mini clean steam generator to supply steam condensate to bioreactor seals.

14. The system of claim 12 wherein the utility area further comprises one or more of: a change over system for gas bottles, a support frame for the gas bottles, a moveable electric clean steam generator and a movable clean in place skid.

15. The system of claim 1 wherein a bioreactor is part of a bioreactor skid that is located in the sterile suite.

16. The system of claim 15 wherein the bioreactor skid further comprises one or more of:
wheels, load cells that can be used for determining vessel volume by weight, a mechanical seal, a temperature control jacket, a condensate manifold, an agitator, an agitator motor, an exhaust vent filter, a spray ball, a gas sparger, and a temperature probe.

17. The system of claim 16 wherein the bioreactor skid further comprises an electrical umbilical cord that can carry one or more of probe signals, load cell signals, a pneumatic valve connection and power to an agitator motor.

18. The system of claim 17 wherein the bioreactor skid further comprises piping connections that can include exhaust gas, addition port, jacket inlet, jacket outlet, WFI or clean steam to the seals, and clean steam or CIP solution to the spray balls, sparge gas and overlay gas.
